(19) United States
(12) Patent Application Publication
(10) Pub. No.: US 2003/0161879 A1
(43) Pub. Date: Aug. 28, 2003

(54) Tablets quickly disintegrating in mouth

(76) Inventors: Shinji Ohmori, Nishinomiya-shi (JP); Yasuo Ohno, Osaka-shi (JP); Tadashi Makino, Ibaraki-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/395,137

(22) Filed: Mar. 25, 2003

Related U.S. Application Data
(62) Division of application No. 09/869,979, filed on Aug. 20, 2001.
Application No. 09/869,979 is a 371 of international application No. PCT/JP00/04081, filed on Jun. 22, 2000.

(30) Foreign Application Priority Data
Jun. 29, 1999 (JP) 183624/1999

(51) Int. Cl. A61K 9/20

(52) U.S. Cl. 424/465

(57) ABSTRACT

Tablets quickly disintegrating in the mouth which comprise a bitter drug ingredient and a bitterness-reducing ingredient composed of an essential oil, a high sweetness-sweetener and/or an acidic phospholipid or its lyso-derivative. When taken even without water, these tablets exhibit little bitterness. Thus, a bitter drug ingredient can be formulated without coating into tablets quickly disintegrating in the mouth.

### FIELD OF THE INVENTION

The present invention relates to tablets quickly disintegrating in the mouth, wherein the bitterness is reduced. More particularly, it relates to tablets quickly disintegrating in the mouth, wherein a bitter drug ingredient can be formulated without coating by compounding a bitterness-reducing ingredient.

### BACKGROUND ART

In recent years, in order to improve QOL (quality of life) of patients, tablets quickly disintegrating in the mouth have received attention as a dosage form that can be taken easily by senior persons or children having a low swallowing ability.

Nevertheless, in the case where a bitter drug ingredient is formulated in tablets quickly disintegrating in the mouth, the tablets are immediately disintegrated in the mouth to expose the bitter drug ingredient in the mouth. In addition in this case, because of being taken without water, tablets quickly disintegrating in the mouth result in perception of a strong bitterness for a longer time than general tablets for internal use, thereby giving a great demerit to the tablets quickly disintegrating in the mouth, which are characterized by easy dosing.

In order to solve the above-mentioned problems, there has been proposed a method that comprises coating a bitter drug ingredient, which is to be formulated in tablets quickly disintegrating in the mouth, with a high molecular compound such as ethyl cellulose, Eudragit, or the like to prevent a direct exposure of said drug ingredient even if said tablets are disintegrated in the mouth (JP 6-5021294 A). However, if the bitter drug ingredient is coated with an aqueous coating agent, a sufficient coating effect cannot be obtained owing to a low strength of the resultant coat. On the contrary, a sufficient coating effect can be obtained by coating using an organic solvent, but there will arise problems involving a residual solvent in the coat, an operational environment at the production such as inhalation of the organic solvent by operators, possible hazards at the operation such as a fire and an explosion, and so on. Also, such a coating has a demerit of requiring an operation for a long time and a facility.

Furthermore, in the case where the coating layer is thickened in order to inhibit the bitterness sufficiently and prevent breakage of the coat upon tabletting, elution of the drug ingredient is delayed, resulting in a preparation from which merely about 30% of the drug ingredient is eluted even after 60 minutes, for example in the dissolution test.

On the other hand, also in the field of granules, powders, liquids and solutions, and the like, technologies to reduce the bitterness have been heretofore developed. There have been proposed a number of technologies in order to reduce the bitterness, for example, a technology of compounding stevia (JP 10-101582 A) or aspartame (JP 2-56416 A), which is classified as a so-called high sweetness-sweetener, that of compounding a combination of a flavor and a sweetener (JP 10-273435 A), that of compounding a sugar alcohol and Thaumatin, one of high sweetness-sweeteners, (for example, JP 10-306038 A), that of using a sugar alcohol (for example, JP 10-53538 A), that of using an essential oil or an essential oil component (for example, JP 5-255126 A), that of using an acidic phospholipid (for examples, JP 62-265234, JP 8-9897 A, JP 7-67552 A, and the like), and so on.

However, they are strictly the technologies of reducing the bitterness for general preparations on the premise that such a preparation is to be taken together with water, whereby, when these technologies were applied, as they are, to tablets quickly disintegrating in the mouth, which were intended to be taken without water, the effect of reducing the bitterness was not sufficient and one had not been able to obtain practical tablets quickly disintegrating in the mouth.

### OBJECTS OF THE INVENTION

The object of the present invention is to provide tablets quickly disintegrating in the mouth without coating a bitter drug ingredient as mentioned above, which can be taken easily with the bitterness being hardly perceived even when taken without water

### SUMMARY OF THE INVENTION

As a result of intensive investigations to solve the above-mentioned problems, the present inventors have found that a combination of an essential oil that has been heretofore known to reduce the bitterness, a high sweetness-sweetener and/or an acidic phospholipid or its lyso-derivative particularly exerts a significant effect to reduce the bitterness, thereby resulting in the completion of the present invention on the basis of such a finding.

That is, the present invention provides:

(1) A tablet quickly disintegrating in the mouth which comprises a bitter drug ingredient and a bitterness-reducing ingredient composed of an essential oil, a high sweetness-sweetener and/or an acidic phospholipid or its lyso-derivative;

(2) The tablet quickly disintegrating in the mouth according to the above (1), wherein the bitterness-reducing ingredient is composed of an essential oil and a high sweetness-sweetener;

(3) The tablet quickly disintegrating in the mouth according to the above (1), wherein the bitterness-reducing ingredient is composed of an essential oil and an acidic phospholipid or its lyso-derivative;

(4) The tablet quickly disintegrating in the mouth according to the above (1), wherein the bitterness-reducing ingredient is composed of essential oil, a high sweetness-sweetener, and an acidic phospholipid or its lyso-derivative;

(5) The tablet quickly disintegrating in the mouth according to the above (1), wherein the bitter drug ingredient is not coated;

(6) The tablet quickly disintegrating in the mouth according to the above (1), wherein the bitter drug ingredient is acetaminophen;

(7) The tablet quickly disintegrating in the mouth according to the above (1), wherein the essential oil is mint oil;

(8) The tablet quickly disintegrating in the mouth according to the above (1), wherein the high sweetness-sweetener is one or two members be selected from stevia and aspartame; and

(9) The tablet quickly disintegrating in the mouth according to the above (1), wherein the acidic phospholipid or its lyso-derivative is soybean lecithin.

### DETAILED DESCRIPTION OF THE INVENTION

Hereinafter, the present invention will be described in detail. In the present specification, the tablets quickly disintegrating in the mouth refer to a solid drug preparation that disintegrates within a time shorter than about 90 seconds, preferably about 60 seconds, without mastication, in the presence of saliva in the mouth.

The bitter drug ingredient to be used in the present invention is exemplified by an ingredient having the bitterness among drug ingredients that are orally administered in general. In particular, preferred examples include unit-dose drug ingredients, which are assumed to be taken inevitably under a water-undrinkable situation, such as an antipyretic-analgesic, an antihistamic agent, an antiallergic agent, a sympathomimetic agent, a parasympathetic blocking agent, a central stimulant, an H₂ blocker, an antacid, an antiphlogistic enzyme preparation, an antiinflammatory agent, a bronchodilator, an antibacterial agent, an antitussive agent, an expectorant agent, a cholinergic blocking agent, an antidiarrheal agent, a sedative hipnotic drug, a cholagogue drug, an antihypertensive agent, a skeletal muscle relaxant, a prophylactic/therapeutic agent against motion sickness or bitter substances among therapeutic agents against osteoporosis, vitamins, crude drugs and the like which are expected to be simply taken daily, which are exemplified by an antipyretic-analgesic such as acetaminophen, ibuprofen, ketoprofen, or the like; an antihistamic agent, an antiallergic agent, such as chlorpheniramine maleate, chlorpheniramine d-maleate, diphenhydramine or its salts, promethazine hydrochloride, isothipendyl hydrochloride, clemastine fumarate, iproheptine hydrochloride, cyproheptadine hydrochloride, diphenylpyraline or its salts, dimethindene maleate, triprolidine hydrochloride, homochlorcyclizine hydrochloride, azelastine hydrochloride, ibudilast, sodium cromoglicate or its salts, oxatomide, amlexanox, carbinoxamine maleate, mequitazine, tranilast, repirinast, emedastine difumarate, ozagrel hydrochloride, tazanolast, pemirolast or its salts, suplatast tosilate, or the like; a sympathomimetic agent such as phenylpropanolamine hydrochloride or the like; a parasympathetic blocking agent such as belladonna (total) alkaloids, isopropamide iodide, or the like, a central stimulant such as caffeine, anhydrous caffeine, or the like; an H₂ blocker such as famotidine, cimetidine, ranitidine hydrochloride, nizatidine, roxatidine acetate hydrochloride, or the like; an antacid such as magnesium carbonate, light anhydrous silicic acid, magnesium aluminometasilicate, synthetic hydrotalcite, or the like; an antiphlogistic enzyme preparation such as lysozyme chloride, serrapeptase, or the like; an antiinflammatory agent such as tranexamic acid or the like; a bronchodilator such as methylephedrine hydrochloride (including the d-form and the dl form), ephedrine hydrochloride, or the like; an antitussive agent such as codeine, codeine phosphate, dihydrocodeine phosphate, dextromethorphan hydrobromide, noscapine, dimemorfan, guaiacolsulfonic acid, guaifenesin, or the like; an expectorant agent such as potassium guaiacosulfonate, bromhexine hydrochloride, or the like; a cholagogue drug such as ursodeoxycholic acid, chenodeoxycholic acid, or the like; an antidiarrheal agent such as loperamide hydrochloride or the like; a sedative hipnotic drug such as bromovalerylurea or the like; an antianxiety agent such as diazepam or the like; a prophylactic/therapeutic agent against motion sickness such as dimenhydrinate or the like; vitamin B₁ family such as thiamine hydrochloride, thiamine mononitrate, bisthiamine nitrate, thiamine disulfide, thiamine dicetylsulfate, dicethiamine hydrochloride, fursultiamine, fursultiamine hydrochloride, octotiamine, cycotiamine, bisibuthiamine, bisbentiamine, prosultiamine, benfotiamine, dibenzoyl thiamine, thiamine pyrophosphate, or the like, vitamin B₂ family such as riboflavin sodium phosphate or the like; vitamin B₆ family such as pyridoxine hydrochloride or the like; calcium pantothenate; vitamin K family such as phytonadione, menatetrenone, or the like; crude drug powder such as Scutellaria root, Phellodendron Bark, or the like and extracts thereof, and so on.

Examples of the drug ingredient, which particularly significantly exerts the effect of reducing the bitterness in the tablets quickly disintegrating in the mouth of the present invention, include acetaminophen, tranexamic acid, noscapine, and bromhexine hydrochloride.

When acetaminophen is used as the bitter drug ingredient, one can select its crystals having the particle size in a range of about 50 to about 800 μm, preferably about 100 to 500 μm. In the case where crystals of acetaminophen have the particle size in the above-described range, the effect of reducing the bitterness is particularly significant.

The amount of the bitter drug ingredient to be formulated is not particularly limited and can be appropriately selected depending on the purpose of the administration, but in general, one can set the range of about 0.01 to 80 parts by weight, preferably about 0.05 to 70 parts by weight, and particularly preferably about 0.1 to 60 parts by weight, per 100 parts by weight of the preparation.

In the tablets quickly disintegrating in the mouth of the present invention, an essential oil as well as a high sweeteness-sweetener and/or an acidic phospholipid or its lyso-derivative are formulated as the bitterness-reducing component.

The examples of the essential oil that can be employed in the present invention include mint oil, eucalyptus oil, cinnamon oil, fennel oil, clove oil, orange oil, lemon oil, and rose oil, preferably mint oil, cinnamon oil, fennel oil, and clove oil, and particularly preferably mint oil.

The amount of the essential oil to be formulated can be appropriately selected depending on the amount of the bitter drug ingredient to be formulated and the bitterness degree thereof together with the amounts of the high sweetness-sweetener and/or the acidic phospholipid or its lyso-derivative to be formulated, but in general, one can set the range of about 0.01 to 10 parts by weight, preferably about 0.02 to 8 parts by weight, and particularly preferably about 0.05 to 5 parts by weight, per 100 parts by weight of the preparation.

The so-called, high sweetness-sweetener to be used in the present invention refers to one having the sweetness of more than several times than that of sugar, preferably more than about 100 times, and is specially exemplified by aspartame, stevia, saccharin, dipotassium glycyrrhizinate, Thaumatin, sucralose, acesulfame-K, or the like and particularly preferably aspartame, stevia, or the like.

The amount of the high sweetness-sweetener can be appropriately selected depending on the amount of the bitter drug ingredient to be formulated and the bitterness degree thereof together with the amounts of the essential oil and the acidic phospholipid or its lyso-derivative to be formulated, but in general, one can set the range of about 0.01 to 20 parts by weight, preferably about 0.05 to 15 parts by weight, and particularly preferably about 0.1 to 10 parts by weight, per 100 parts by weight of the preparation.

The acidic phospholipid or its lyso-derivative to be used in the present invention has been known to possess, as itself, the action to reduce the bitterness (JP 7-67552 A) and is an acidic phospholipid such as phosphatidylserine, phosphatidic acid, phosphatidylinositol, phosphatidylglycerol, cardiolipin, or the like as well as a lyso-derivative thereof such as lyso-phosphatidylserine, lyso-phosphatidic acid, lyso-phosphatidylinositol, lyso-phosphatidylglycerol, or the like. These acidic phospholipids or their lyso-derivatives can be obtained as soybean lecithin, egg yolk lecithin, and the like by extraction and separation from a variety of animals and plants according to the method described in JP 8-9896 A. Also, as for the acidic phospholipid or its lyso-derivative to be used in the present invention, one that is commercially available, for example, as Benecoat BMI-60 (the trade name of Kao) can be used.

The amount of the acidic phospholipid or its lyso-derivative to be formulated can be appropriately selected depending on the amount of the bitter drug ingredient and the bitterness degree thereof to be formulated together with the amounts of the essential oil and the high sweetness-sweetener to be formulated, but in general, one can set the range of about 0.01 to 20 parts by weight, preferably about 0.05 to 15 parts by weight, and particularly preferably about 0.1 to 10 parts by weight, per 100 parts by weight of the preparation.

The tablets quickly disintegrating in the mouth of the present invention can be produced according to a conventional method for producing tablets quickly disintegrating in the mouth, except that the above-mentioned bitter drug ingredient, which is an essential constitutive ingredient in the present invention, the essential oil as well as the high sweetness-sweetener and/or the acidic phospholipid or its lyso-derivative are compounded. Specifically, they can be produced according to a method, for example that described in JP 10-182436 A, namely a method for compounding erythritol, crystalline cellulose and an disintegrating agent in addition to the above-mentioned essential constitutive ingredients of the present invention (see Examples 1 to 5 hereinafter).

Also, in the tablets quickly disintegrating in the mouth of the present invention, a variety of excipients to be used in the production of a conventional solid preparation can be compounded, as far as they do not interfere with the advantages of the present invention.

In the case where the essential oil, the high sweetness-sweetener and/or the acidic phospholipid or its lyso-derivative are compounded at the same time to the tablets quickly disintegrating in the mouth of the present invention, porous calcium silicate may be added in order to prevent a lowering of the hardness of the tablets. As for the amount of porous calcium silicate to be formulated in this case, it is preferable to formulate the silicate in a range of about 0.5 to 30 parts by weight per 100 parts by weight of the tablets quickly disintegrating in the mouth of the present invention. Porous calcium silicate to be used here is commercially available easily as Florite RE (the trade name of Tokuyama Soda).

The essential oil (for example, mint oil) formulated in the present invention is evaporated during a storage period, if it is allowed to stand without being packaged. When the amount of the essential oil (for example, mint oil) becomes smaller, the effect of the bitterness masking in the present invention decreases, resulting in giving perception of the bitterness. Also, storage of the tablets of the present invention with exposure under a high humidity induces a hygroscopic lowering in the hardness. Thus, a preferred packaging form of the tablets quickly disintegrating in the mouth of the present invention is a closed container such as a strip package, a blister package, a package in a bottle, of which particularly preferred is a packaging where a aluminum-strip package or a blister package having a high hermeticity is enclosed in a pouch (for example, of aluminum foil or a film laminated with polyethylene).

The present invention is illustrated in more detail by the following examples, comparison examples, and comparison test.

### EXAMPLE 1

### Essential Oil+Highly Sweetness-Sweetener+Soybean Lecithin+Porous Calcium Silicate

In a mortar were placed 30 g of erythritol, 8.41 g of crystalline cellulose (Ceolus KG801), 2.45 g of Crospovidone, 1.0 g of soybean lecithin (Benecoat BMI-60), 2 g of porous calcium silicate (Florite RE), 0.25 g of stevia, and 0.5 g of aspartame and after addition of 100 μl of mint oil, the resulting mixture was pestled. Into the thus-obtained powder were mixed 15 g of acetaminophen and 0.3 g of magnesium stearate and the resulting mixture was subjected to tabletting with an autograph (AG-5000B, Shimazu Seisakusyo) by using a 13-mm mill and a flat pounder at a tabletting pressure of 1400 kg/pounder to prepare tablets each weighing 600 mg.

### EXAMPLE 2

### Essential Oil+High Sweetness-Sweeteneragent+Soybean Lecithin

In a mortar were placed 30 g of erythritol, 9.91 g of crystalline cellulose (Ceolus KG801), 2.45 g of Crospovidone, 1.5 g of soybean lecithin (Benecoat BMI-60), 0.25 g of stevia, and 0.5 g of aspartame and after addition of 100 μl of mint oil, the resulting mixture was pestled. Into the thus-obtained powder were mixed 15 g of acetaminophen and 0.3 g of magnesium stearate and the resulting mixture was subjected to tabletting with an autograph (AG-5000B, Shimazu Seisakusyo) by using a 13-mm mill and a flat pounder at a tabletting pressure of 1400 kg/pounder to prepare tablets each weighing 600 mg.

### EXAMPLE 3

### Essential Oil+High Sweetness-Sweetener

In a mortar were placed 15 g of acetaminophen, 39.7 g of erythritol, 7.16 g of crystalline cellulose (Ceolus KG801), 2 g of Crospovidone, 0.25 g of stevia, and 0.5 g of aspartame and after addition of 100 μl of mint oil, the resulting mixture was pestled. Then, an appropriate amount of purified water was added and after kneading, the resulting mixture was dried under vacuum at 40° C. for 16 hours. After the thus-obtained, granular powder was sized to 16 meshes, 0.3 g of magnesium stearate was mixed to 6.47 g of the sized powder and the resulting mixture was subjected to tabletting with an autograph (AG-5000B, Shimazu Seisakusyo) by using a 13-mm mill and a flat pounder at a tabletting pressure of 1400 kg/pounder to prepare tablets each weighing 650 mg.

### EXAMPLE 4

### Essential Oil+High Sweetness-Sweetener+Soybean Lecithin+Porous Calcium Silicate

In a mortar were placed 30 g of erythritol, 8.41 g of crystalline cellulose (Ceolus KG801), 2.45 g of Crospovidone, 1.0 g of soybean lecithin (Benecoat BMI-60), 2 g of special calcium silicate (Florite RE), 0.25 g of stevia, and 0.5 g of aspartame and after addition of 100 μl of mint oil, the resulting mixture was pestled. Into the thus-obtained powder were mixed 12.5 g of tranexamic acid and 0.3 g of magnesium stearate and the resulting mixture was subjected to tabletting with an autograph (AG-5000B, Shimazu Seisakusyo) by using a 13-mm mill and a flat pounder at a tabletting pressure of 1400 kg/pounder to prepare tablets each weighing 575 mg.

### EXAMPLE 5

### Essential Oil+High Sweetness-Sweetener+Porous Calcium Silicate

A mixture of 900 g of erythritol, 160.5 g of crystalline cellulose (Ceolus KG801), 72 g of Crospovidone, 18 g of porous calcium silicate (Florite RE), 0.36 g of yellow-No. 5 aluminum lake is subjected to granulation in a fluidized-bed granulator (FD-3SN, POWREX) with spraying purified water, followed by fluidized-bed drying, to obtain a granulated powder. This powder is sized by using a power mill (Showa Kikai Kohsakusyo) to obtain a sized powder. Separately, in a mortar are placed 54.98 g of crystalline cellulose (Ceolus KG801), 2.5 g of stevia, and 5 g of aspartame and after addition of 1 ml of mint oil, the resulting mixture is pestled to obtain a mint oil-triturated powder. Into this mint oil-triturated powder are mixed 383.62 g of the sized powder, 150 g of acetaminophen, and 3 g of magnesium stearate to obtain a mixed powder. The resulting mixed powder is subjected to tabletting with a rotary-type tabletting machine by using a 13-mm mill and a flat pounder at a tabletting pressure of 1400 kg/pounder to prepare tablets each weighing 600 mg.

### COMPARISON EXAMPLE 1

### Highly Sweet, Sweetening Agent

A mixture of 15 g of acetaminophen, 30 g of erythritol, 1.15 g of crystalline cellulose (Ceolus KG801), 2.5 g of Crospovidone, 0.25 g of stevia, and 0.5 g of aspartame was subjected to tabletting with an autograph (AG-5000B, Shimazu Seisakusyo) by using a 13-mm mill and a flat pounder at a tabletting pressure of 1400 kg/pounder to prepare tablets each weighing 600 mg.

### COMPARISON EXAMPLE 2

### Essential Oil

In a mortar were placed 30 g of erythritol, 12.16 g of crystalline cellulose (Ceolus KG801), and 2.45 g of Crospovidone and after addition of 100 μl of mint oil, the resulting mixture was pestled. Into the thus-obtained powder were mixed 15 g of acetaminophen and 0.3 g of magnesium stearate and the resulting powder was subjected to tabletting with an autograph (AG-5000B, Shimazu Seisakusyo) by using a 13-mm mill and a flat pounder at a tabletting pressure of 1400 kg/pounder to prepare tablets each weighing 600 mg.

Comparison Test

The tablets quickly disintegrating in the mouth of the present invention obtained in the above-described Examples 1 to 4 as well as the tablets quickly disintegrating in the mouth obtained in Comparison Examples 1 and 2 were subjected to tests for the hardness and the time for disintegrating in the mouth, followed by evaluation of the bitterness, respectively. For the evaluation of the bitterness, the following scores were employed to evaluate separately the bitterness at 10 seconds after taking the tablets and the bitterness at 60 seconds after taking the tablets.

The Scores for Evaluating the Bitterness

The bitterness is hardly perceived: 0

The bitterness is slightly perceived: 1

The bitterness is perceived: 2

The bitterness is strongly perceived: 3

The case where the score is 0 or 1 refers to the bitterness degree involving no problem in taking the tablets and the case where the score is 2 or 3 refers to the bitterness degree involving difficulty in taking the tablets.

The results are shown in the following Table 1.

TABLE 1

| | | | | | | Comparison | |
|---|---|---|---|---|---|---|---|
| | Example | | | | | Example | |
| | 1 | 2 | 3 | 4 | 5 | 1 | 2 |
| Hardness | 6.1 | 2.3 | 5.7 | 7.4 | 4.5 | 7.9 | 4.3 |
| | kg | kg | kg | kg | kg | kg | kg |
| Disintegration | 25 | 40 | 20 | 35 | 20 | 20 | 15 |
| time in | sec. | sec. | sec. | sec. | sec. | sec. | sec. |
| mouth | | | | | | | |
| Bitterness | | | | | | | |
| evaluation | | | | | | | |
| 10 sec. | 0 | 0 | 0 | 0 | 0 | 2 | 2 |
| after | | | | | | | |
| takeing | | | | | | | |
| 60 sec. | 0 | 0 | 0 | 0 | 0 | 3 | 3 |
| after | | | | | | | |
| takeing | | | | | | | |

According to the above-described Table 1, the effect of reducing the bitterness in the quickly disintegrating in the mouth where only either of the essential oil or the highly sweetness-sweeteners was formulated (Comparison Examples 1 or 2) is not sufficient, whereas the bitterness in the tablets quickly disintegrating in the mouth of the present invention (Examples 1 to 5) was reduced to a degree involving no problem in the actual taking. Particularly, an excellent effect in the tablets quickly disintegrating in the mouth of the present invention is evident from the point that the bitterness is reduced at 60 seconds after the taking.

### EXAMPLE 6

The tablets obtained in Example 5, which were packaged in aluminum strips and stored for 6 months at 40° C. and a relative humidity of 75%, were subjected to evaluation of the hardness, the time for disintegrating in the mouth, and the bitterness. The evaluation of the bitterness is carried out in a manner similar to that in Table 1.

### COMPARISON EXAMPLE 3

The tablets obtained in Example 5, which were placed in a glass bottle and stored for 6 months at 40° C. and a relative humidity of 75% with the cap being opened, were subjected to evaluation of the hardness, the time for disintegrating in the mouth, and the bitterness. The evaluation of the bitterness is carried out in a manner similar to that in Table 1.

Table 2 shows the results on the evaluation in Example 6 and Comparison Example 3.

TABLE 2

| | Example 6 | Comparison Example 3 |
|---|---|---|
| Hardness | 4.5 kg | 2.1 kg |
| Disintegration time | 20 sec. | 18 sec. |
| in mouth | | |
| Bitterness evaluation | | |
| 10 seconds after | 0 | 2 |
| takeing | | |
| 60 seconds after | 0 | 3 |
| takeing | | |

As evident from Table 2, it is preferable that the tablets quickly disintegrating in the mouth of the present invention are stored in the packaging form having a high hermeticity such as the aluminum strip package.

As described hereinabove, the tablets quickly disintegrating in the mouth of the present invention can be taken without water with the bitterness being hardly perceived, despite that the bitter drug ingredient is not coated. Therefore, they provide extremely useful preparations in the field of medical care in the point that not only they can be produced at a lower cost as compared to the case where the drug ingredient is coated, but also. any necessary drug ingredient can be taken at any time and in any location.

What is claimed is:

1. A tablet quickly disintegrating in the mouth which comprises a bitter drug ingredient and a bitterness-reducing ingredient composed of an essential oil, a high sweetness-sweetener and/or an acidic phospholipid or its lyso-derivative.

2. The tablet quickly disintegrating in the mouth according to claim 1, wherein the bitterness-reducing ingredient is composed of an essential oil and a high sweetness-sweetener.

3. The tablet quickly disintegrating in the mouth according to claim 1, wherein the bitterness-reducing ingredient is composed of an essential oil and an acidic phospholipid or its lyso-derivative.

4. The tablet quickly disintegrating in the mouth according to claim 1, wherein the bitterness-reducing ingredient is composed of essential oil, a high sweetness-sweetener, and an acidic phospholipid or its lyso-derivative.

5. The tablet quickly disintegrating in the mouth according to claim 1, wherein the bitter drug ingredient is not coated.

6. The tablet quickly disintegrating in the mouth according to claim 1, wherein the bitter drug ingredient is acetaminophen.

7. The tablet quickly disintegrating in the mouth according to claim 1, wherein the essential oil is mint oil.

8. The tablet quickly disintegrating in the mouth according to caim 1, wherein the high sweetness-sweetener is one or two members be selected from stevia and aspartame.

9. The tablet quickly disintegrating in the mouth according to claim 1, wherein the acidic phospholipid or its lyso-derivative is soybean lecithin.
